(19) United States
(12) Patent Application Publication
(10) Pub. No.: US 2023/0320570 A1
(43) Pub. Date: Oct. 12, 2023

(54) BITE BLOCK WITH SHIELD OR MASK

(71) Applicant: Mayo Foundation for Medical Education and Research, Rochester, MN (US)

(72) Inventors: Elizabeth Rajan, Rochester, MN (US); Jad P. AbiMansour, Rochester, MN (US)

(21) Appl. No.: 18/023,138

(22) PCT Filed: Aug. 17, 2021
(86) PCT No.: PCT/US2021/046254

§ 371 Date: Feb. 24, 2023

Related U.S. Application Data
(60) Provisional application No. 63/069,952, filed on Aug. 25, 2020.

(51) Int. Cl. A61B 1/00; A61B 90/16; A61B 90/00

(52) U.S. Cl. CPC A61B 1/00131; A61B 1/00154; A61B 90/16; A61B 90/05

(57) ABSTRACT

This document describes devices for reducing risks to healthcare staff from infectious agents. For example, this document describes a bite block with a shield element or a mask that can reduce risk of infectious or noxious agents exposure to healthcare staff.

### CROSS-REFERENCE TO RELATED APPLICATIONS

This application claims the benefit of U.S. Provisional Application Ser. No. 63/069,952, filed Aug. 25, 2020. The disclosure of the prior application is considered part of (and is incorporated by reference in) the disclosure of this application.

### BACKGROUND

### 1. Technical Field

This document relates to devices for reducing risks to healthcare staff from infectious agents. For example, this document relates to a bite block with a shield and a bite block with a mask that can reduce risk of Covid-19 or other infectious agents exposure to healthcare staff

### 2. Background Information

When performing endoscopy, healthcare staff, such as GI endoscopists and nurses, face risks of droplet or aerosolized transmission of infectious agents such as viruses (e.g., Covid-19) from patients. Transmission occurs through the oral and nasal route particularly when patients cough, gag or exhale during endoscopy.

Bite blocks are placed in the patient's mouth for upper/oral exams. The bite block has a central opening for endoscope passage and two side openings to secure the bite block in position with a strap. The side openings can also be used for passage of accessories such as suction, supplemental oxygen or endotracheal tube. These unsealed side openings create risk for droplet and aerosolized transmission.

### SUMMARY

This document describes devices for reducing risks to healthcare staff from infectious agents. For example, this document describes a bite block with a shield and a bite block with a mask that can reduce risk of Covid-19 or other infectious agents exposure to healthcare staff.

In one aspect, this disclosure is directed to a shielded bite block. Such a shielded bite block can include a shield element made of material comprising synthetic polymer fibers, and a bite block including a frame and a bite member defining a passage. The shield element is attached to the bite block or configured to be attached to the bite block.

Such a shielded bite block may optionally include one or more of the following features. The shield element may define an opening aligned with the passage. The shielded bite block may also include a removable or openable cover to seal the opening. The shield element may define one or more additional openings with respective covers that seal the one or more additional openings and that are removable or openable.

In another aspect, this disclosure is directed to a bite block mask. Such a bite block mask can include a face mask shaped and sized for covering a mouth and nose of a patient, and a bite block attached to the face mask. The bite block mask defines a first opening that extends though the face mask and the bite block.

Such a bite block mask may optionally include one or more of the following features. The first opening may be configured for passing an endoscope therethrough. The bite block mask may also include an air filter for exhaled air. The air filter may be attached to the face mask. The face mask may define one or more additional openings therethrough. The bite block mask may also include one or more straps extending from the face mask and configured for securing the bite block mask to the patient. The bite block mask may also include a compliant seal extending along at least a portion of a periphery of the face mask.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 shows perspective view an example shielded bite block in accordance with some embodiments provided herein.

FIG. 2 shows a front view of the shielded bite block of FIG. 1.

FIG. 3 shows a back view of the shielded bite block of FIG. 1.

FIG. 4 shows another front view of the shielded bite block of FIG. 1

FIG. 5 shows a front view of an example bite block with mask in accordance with some embodiments.

FIG. 6 shows a back view of the bite block with mask FIG. 5.

FIG. 7 shows a perspective back view of the bite block with mask FIG. 5.

Like reference numbers represent corresponding parts throughout.

### DETAILED DESCRIPTION

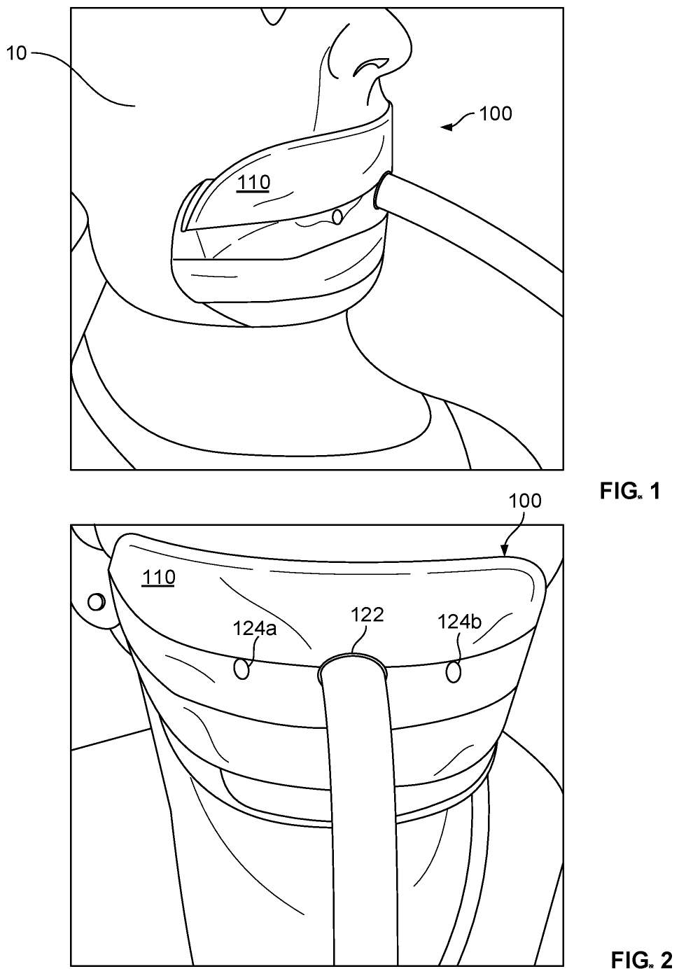

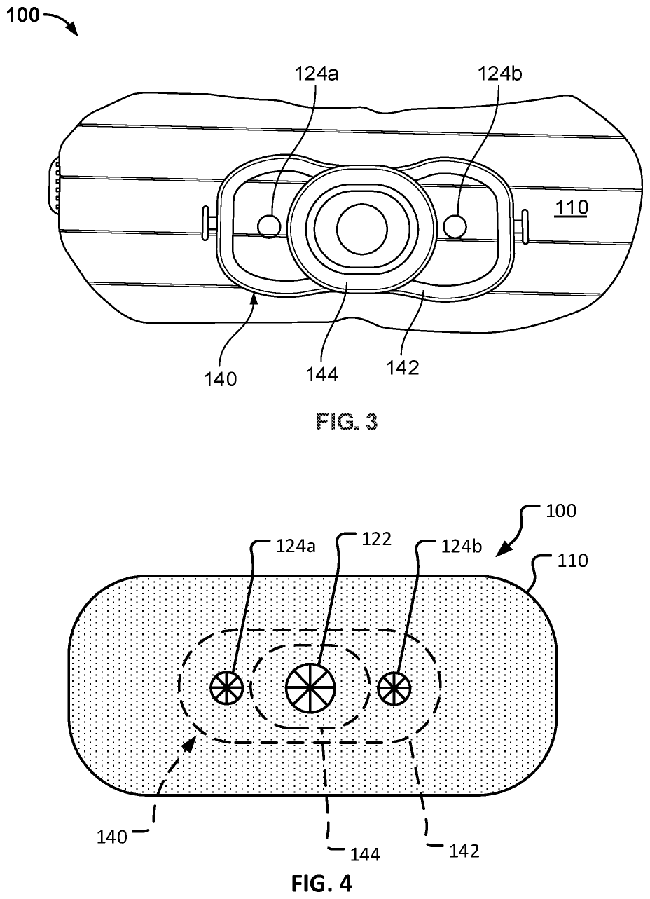

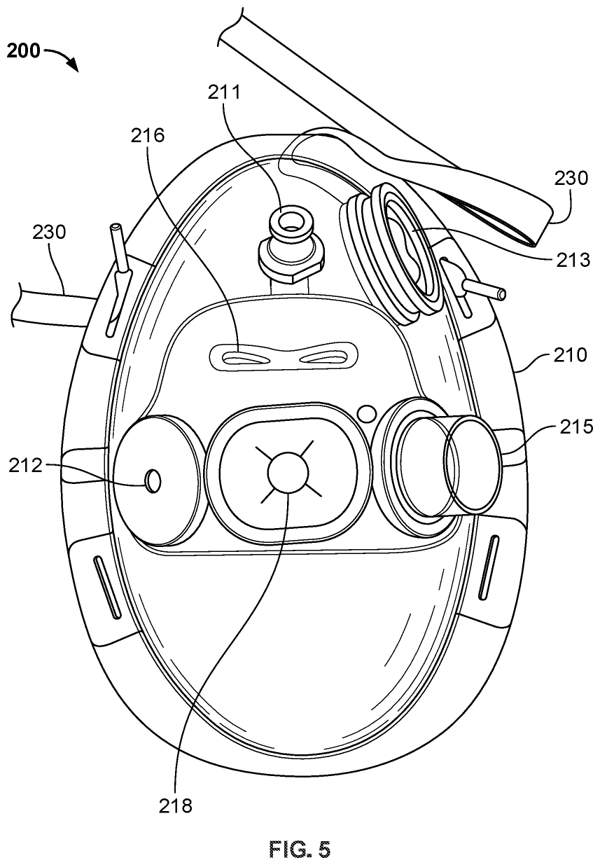

As shown in FIGS. 1-4, a shielded bite bock 100 can be used by a patient 10 during procedures such as, but not limited to, upper GI endoscopy (including ERCP and EUS), bronchoscopy (including EBUS), laryngoscopy, awake intubations, and perioperative anesthesia.

The shielded bite bock 100 includes a shield element 110 and a bite block 140. In some embodiments, the shield element 110 is permanently attached/affixed to the bite block 140. Alternatively, in some embodiments the shield element 110 is releasably attachable to the bite block 140, such as by using an adhesive, clips, magnets, barbs, or other types of releasably attachment means.

In some embodiments, the shielded bite bock 100 is configured to be used by the patient 10 such that the top of the shield element 110 is just below the nose and the bottom of the shield element 110 is on the chin of the patient 10. Alternatively, in some embodiments the shield element 110 can be configured to cover the nose of the patient 10.

In some embodiments, one or more elastic straps or tie straps are included to help secure the shielded bite bock 100 to the patient 10. In some embodiments, one or more malleable members can be included in or on the shield element 110 whereby the shield element 110 can be custom shaped to conform to the facial topography of the patient 10.

The shielded bite bock 100 includes the shield element 110. In some embodiments, one or more portions of the shield element 110 is/are made of the same types of materials used for respirator masks (e.g., N95 masks). For example, in some embodiments the shield element 110 is made of synthetic polymer fibers such as, but not limited to, polypropylene. The shield element 110 should be comprised of a material that is essentially impervious to the emissions of infectious agents.

The bite block 140 includes a frame 142 and a bite member 144. The bite member 144 is configured to be held within the mouth of the patient 10 (e.g., by biting). A passage is defined through the bite member 144. The passage is used for endoscope access, for example.

In some embodiments, the shield member 110 defines an opening 122 in alignment with the opening of the bite member 144. In particular embodiments, the opening 122 includes a removable or openable cover/seal (to be removed or opened as needed to pass through an instrument such as an endoscope, for example). This opening/seal may have reinforced teeth-like configuration, or slits or flaps, or an expandable band that allows easy endoscope passage while providing a more secure seal.

In some embodiments, the shield member 110 also defines one or more ancillary openings 124a and 124b. These openings 124a-b can also include a removable or openable cover/seal (to be removed or opened as needed to pass through an instrument such as a suction device, for example). These openings 124a-b have a cover/seal that can be closed when not in use.

Optional features and/or alternatives include a one-way valve mechanism for more secure closure. Patients can still breathe through the material and/or via nasal cannula. This shield may be a stand-alone product placed over the existing bite block or in another embodiment this shield may be incorporated as an integral part of the bite block with the described features. In some embodiments, the shield element 110 and bite block 140 can be transparent allowing for visualization. In some embodiments, the bite block 140 can have one central opening 122 without the two side openings 124a-b. In some embodiments, the bite block 140 can be “strapless,” e.g., being secured in place using the shield and/or an adhesive and not a strap.

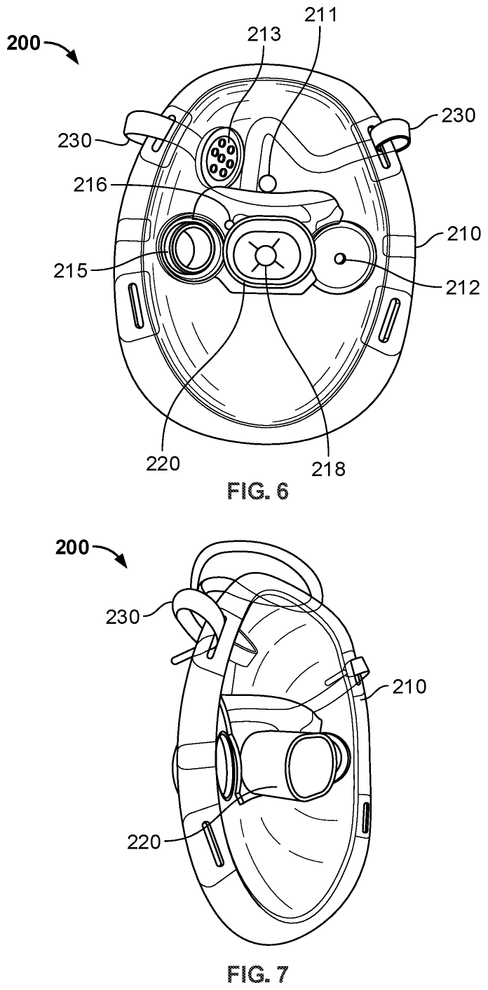

FIGS. 5-7 illustrate an example bite block mask 200. In brief, the bite block mask 200 includes a bite block with a bite member 220 that is incorporated into a face mask 210 as an integral part of the bite block mask 200.

The bite block mask 200 is a single device that provides the combined function of an oxygen-delivery face mask and a bite block for endoscopy, while providing safety to healthcare staff against infectious pathogens. Performing endoscopy through a hole in the standard oxygen mask and then advancing the endoscope to a standard bite block creates a space or gap between the endoscopist's hand and patient's mouth which impedes proper endoscope manipulation by the physician and successful exam. With this new bite block mask 200 combination device, the bite block 220 is attached to the face mask 210 and is positioned directly within the patient's mouth to ensure there is no space between the oxygen mask 210 and bite block 220 allowing for the endoscopist to be perform the procedure with ease, and in the familiar manner.

The face mask 210 is configured (e.g., shaped and sized) for covering a mouth and nose of a patient, and for conforming to a face of the patient. In the depicted embodiment, the face mask 210 defines or includes one or more ports/openings and/or other features. The ports/openings and/or other features can be combined in any combination. Not all of the ports/openings and/or other features are required in various embodiments. The depicted embodiment includes an oxygen delivery port 211, a capnography opening 212 that can be used for the measurement of end tidal CO2, a port for an endotracheal tube 215, one or more nasal openings 216 for delivery of oxygen through a nasal cannula, and an opening 218 in alignment with the opening of the bite member 220. The opening 218 is used for endoscope access, for example. The opening 218 could also be used for accessories (e.g., for an oral suction catheter). This opening 218 may have reinforced teeth-like configuration or slits or flaps that allows easy endoscope passage while providing a more secure seal. Each of the ports/openings and features is individually optional. In some embodiments, the oxygen delivery port 211 and capnography opening 212 may be combined as a single port.

The face mask 210 can include an adjustable nose clip to secure/conform the mask 210 to the nasal bridge of the patient. Such an adjustable nose clip may be made of malleable material such as metal (e.g., aluminum, stainless steel, etc.).

The bite block mask 200 can also include one or more straps 230 to secure the face mask to the patient's face. In some embodiments, no straps 230 are included and the way the bite block mask 200 is maintained in engagement with the patient is solely by the patient biting on the bite member 220.

In some embodiments, the margins of the face mask 210 may be cushioned with a compliant seal for comfort and sealing against the patient's face. The face mask 210 can be transparent allowing for visualization. The face mask 210 may be made of material(s) such as plastic such as PVC and/or silicone.

The bite block includes the bite member 220 configured to be held within the mouth of the patient. The bite member 220 can be integral to the face mask 210. The bite member 220 may or may not have a frame. Such a frame may be conformed to the shape of the bite member 220 (e.g., solid oval frame around the bite member 220).

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A shielded bite block comprising:
a shield element made of material comprising synthetic polymer fibers; and
a bite block including a frame and a bite member defining a passage,
wherein the shield element is attached to the bite block or configured to be attached to the bite block.

2. The shielded bite block of claim 1, wherein the shield element defines an opening aligned with the passage.

3. The shielded bite block of claim 2, further comprising a removable or openable cover to seal the opening.

4. The shielded bite block of claim 1, wherein the shield element defines one or more additional openings with respective covers that seal the one or more additional openings and that are removable or openable.

5. A bite block mask comprising:
a face mask shaped and sized for covering a mouth and nose of a patient; and
a bite block attached to the face mask,
wherein the bite block mask defines a first opening that extends though the face mask and the bite block.

6. The bite block mask of claim 5, wherein the first opening is configured for passing an endoscope therethrough.

7. The bite block mask of claim 6, further comprising an air filter for exhaled air, wherein the air filter is attached to the face mask.

8. The bite block mask of claim 7, wherein the face mask defines one or more additional openings therethrough.

9. The bite block mask of claim 5, further comprising one or more straps extending from the face mask and configured for securing the bite block mask to the patient.

10. The bite block mask of claim 5, further comprising a compliant seal extending along at least a portion of a periphery of the face mask.
